(19) United States
(12) Patent Application Publication
(10) Pub. No.: US 2015/0202013 A1
(43) Pub. Date: Jul. 23, 2015

(54) SHAFT FOR MEDICAL INSTRUMENTS, COMPRISING MOVABLE SECTIONS

(71) Applicant: Richard Wolf GmbH, Knittlingen (DE)

(72) Inventors: Elmar Teichtmann, Bretten (DE); Frank Wehrheim, Bretten (DE)

(21) Appl. No.: 14/416,910

(22) PCT Filed: Jul. 24, 2013
(86) PCT No.: PCT/EP2013/065616

§ 371 Date: Jan. 23, 2015

(30) Foreign Application Priority Data
Jul. 24, 2012 (DE) 10 2012 212 997.9

(51) Int. Cl. A61B 19/00; A61B 17/00

(52) U.S. Cl. CPC A61B 19/22; A61B 19/2203; A61B 17/00234; A61B 2017/00314; A61B 2017/00323; A61B 2019/2238; A61B 2019/2242

(57) ABSTRACT

A medical instrument includes a shaft that includes at least two adjacent shaft sections which are movable relative to one another. The at least two movable shaft sections are engaged with each other so as to roll off one another in a frictionally locking manner or so as mesh with each other via teeth.

### CROSS REFERENCE TO RELATED APPLICATIONS

This application is a United States National Phase Application of International Application PCT/EP2013/065616 filed Jul. 24, 2013 and claims the benefit of priority under 35 U.S.C. §119 of German Patent Application DE 10 2012 212 997.9 filed Jul. 24, 2012, the entire contents of which are incorporated herein by reference.

### FIELD OF THE INVENTION

The present invention relates to a medical instrument with a shank, which comprises at least two adjacent shank sections which are movable relative to one another.

### BACKGROUND OF THE INVENTION

Medical instruments are known, in particular endoscopic instruments which comprise a shank which is designed in a bendable manner in at least one region. Thereby, shanks are known, which comprise individual pivot joints or however movable shanks which comprise a multitude of shank sections which are movable to one another.

In recent times, there are attempts at applying robot systems for operations. With these, it is absolutely necessary for the control to know the exact instrument position. With shanks which comprise several shank sections which can be angled or bent to one another and which are pivoted via pull cables however, there exists the problem however that individual shank sections do not execute a precisely defined movement to one another. In contrast, the movement depends on how the sections bear on adjacent tissue for example. Such a system is thus unsuitable for a robotic operating system.

### SUMMARY OF THE INVENTION

With regard to this problem, it is an object of the invention, to improve a medical instrument with a shank comprising at least two adjacent shank sections which are movable to one another, to the extent that the shank sections are pivoted to one another in a defined manner.

The medical instrument according to the invention in the known manner comprises at least one preferably hollow shank which comprises at least two adjacent shank sections which can be moved to one another. I.e. the two shank sections can be pivoted to one another, which is to say with respect to one another. According to the invention, the at least two movable shank sections are designed such that they are either engaged with one another in a positively meshing manner via toothings, or are frictionally engaged with one another, so that they roll on one another in a slip-free manner. I.e. the two movable shank sections, with the pivot movement, roll on one another in a slip free manner, in the manner of a cog gear or frictionally, and are always engaged positively or non-positively with one another in a defined manner. I.e. the two shank sections always carry out a defined movement relative to one another which predefined by the toothing or frictional fit, if for example they are actuated into a deflection by way of pull elements.

Particularly preferably, the shank comprises a plurality of shank sections which are movable to one another, of which in each case two shank sections which are adjacent to one another, rolling on one another via toothings or frictionally, are meshingly engaged with one another. Thus, several or many joint locations between the individual shank sections which are adjacent one another or are neighboring one another are created, wherein a defined rolling movement of the shank sections adjacent one another, to one another, is always achieved in these joint locations due to the non-positive or positive engagement. Such a defined movement renders such a flexible instrument useable also for surgery based on robotics, since such a shank can be moved or controlled, such that the movements are always defined such that the position of the shank and in particular of the distal end of the shank is known to the control.

The toothings are preferably designed such that two adjacent shank sections at their face ends which lie opposite one another are provided with corresponding toothings which are engaged with one another such that the two shank sections can be pivoted to one another. This pivot movement in particular can be effected by way of a rolling of the shank sections on one another or in the toothings which are engaged with one another.

The shank sections, in the case that a frictional engagement of the shank sections is envisaged, each comprise friction or engagement surfaces which frictionally bear on one another, i.e. contact surface or friction surfaces which correspond to one another and are frictionally engaged with one another such that the two shank sections can be pivoted to one another, are formed on two adjacent shank sections at their face ends which lie opposite one another. Thereby, the friction surfaces roll on one another in a slip-free manner. The friction surfaces which correspond to one another and which bear on one another can comprise roughened or elastic surface structures which ensure a frictional engagement and slip-free rolling.

At least individual toothings or friction surfaces are designed in an arched manner, in order to permit this. I.e. at least one of the two toothings or friction surfaces which are engaged with one another is designed in an arched or arcuate manner. Preferably, two toothings meshing with one another or frictions surfaces which are in engagement are designed in an arched manner, so that a rolling movement on one another is effected.

Thereby, the toothings which mesh with one another or friction surfaces rolling on one another further preferably have the same radius of curvature at the face sides of two adjacent shank sections which lie opposite one another. A uniform bending is thus achieved.

Furthermore, the toothings which mesh with one another or friction surfaces which roll on one another are preferably curved in opposite directions, at the face ends of two adjacent shank sections which are opposite one another. I.e. there are two arched outer toothings which each form a section of a cog, or arched wheel sections. The arched shape thereby preferably corresponds to a circular arc shape as described above. Thus, an engagement of the two shank sections which are distanced to one another is achieved in the manner of a cog gear or a frictional rolling in the manner of a friction gear.

Further preferably, the adjacent shank sections at their face sides which face one another in each case on two diametrically opposite sides comprise a toothing or friction surface. The toothings or friction surfaces thus preferably extend in at least two planes which are parallel to one another and to the longitudinal axis of the shank section and which form the pivot planes, in which the two shank sections which are parallel to one another can pivot to one another. The toothings or friction surfaces thereby preferably extend in planes which are arranged tangentially or in a chord-like manner to the outer wall of the shank section.

As a whole, the toothings or friction surfaces thus preferably extend in an arched manner about the same axis, at the two diametrically opposite sides. Thereby, as described, they have a circular arc shape. Correspondingly, an identically designed toothing or friction surfaces are situated on the opposite side of an adjacent shank section.

Particularly preferably, the shank comprises at least three shank sections which are movable to one another, of which the respective adjacent shank sections are engaged with one another meshingly via toothings or frictionally in a manner rolling on one another. Thereby, the toothings or friction surfaces can be designed in the previously described manner. With this preferred embodiment, further preferably the first and the second shank section are movable to one another in a first pivot plane, and the second and third shank section are movable to one other in a second pivot plane. I.e. the pivot planes between the individual shank sections alternate with one another, so that movability to one another in different direction is achieved. Preferably, the first and the second pivot plane extend normally to one another, so that the individual pivot planes alternate in a manner offset to one another in each case by 90° with respect to the longitudinal axis of the shank A deflection of the shank in all spatial directions is possible by way of this, by way of the pivot movements being combined or superimposed in the two directions. Preferably, the extension planes, in which the arched toothings or friction surfaces are situated, are arranged in each case offset by 90° to one another from one interface to the next, in order to achieve this alternating alignment of the pivot planes between the individual shank sections. I.e. the arched toothings or friction surfaces between the first and the second shank section are arranged in planes parallel to the first pivot plane, whereas the arched toothings or friction surfaces between the second and the third shank section are arranged parallel to the second pivot plane. This then accordingly continues in the case of more than three shank sections.

The toothings or friction surfaces are thereby designed in an arched manner, and the toothings or friction surfaces between the first and the second section are preferably arcuate about axes, i.e. particularly preferably about the same axis, which extend normally to the axes or axis, about which the toothings or frictions surfaces between the second and the third section are arcuate. These axes, about which the toothings or friction surfaces are arcuate, extend normally to the respective pivot planes, in which the pivot movements between the two adjacent shanks sections are carried out.

According to a particularly preferred embodiment, the at least two movable shank sections are meshingly engaged with one another via toothings and simultaneously roll on one another via arched contact surfaces. This means that in each case, at least one arched toothing and at least one arched contact surface are formed on the two shank sections, on sides facing one another. Thereby, the toothings engage into one another and the arched contact surfaces which face one another, bear on one another. This design has the advantage that the toothings, via the arched contact surfaces, are held at a defined distance to one another on pivoting, which means that a constant axis distance between the pivot axes of the two shank sections is kept to. This is defined by the arched contact surfaces.

Further preferably, such a design consisting of arched toothings and arched contact surfaces which ensure a constant axis distance, is formed in each case between the shank sections adjacent one another, in the case that more than two sections are provided. Thereby, the toothings can be designed and arranged in the previously described manner.

Further preferably, a lateral guide is simultaneously provided, which prevents a displacement of shank sections which are adjacent one another, in the direction of the pivot axis. Such a lateral guide can be formed by a wall or flank which is adjacent to the arched contact surface which means extends transversely to the contact surface. For example, the heads of the teeth can project in the radial direction beyond the adjacent contact surface, so that side flanks arise between the teeth, on which side flanks the projecting teeth of an oppositely lying shank section abut given a lateral movement, so that this movement is limited or prevented.

Further preferably, each shank section comprises at least one arched contact surface and a toothing, wherein the arched contact surface is usefully arcuate parallel to the toothing. One succeeds in the axis distance between the shank sections being kept constant in every angular position on pivoting by way of this. Particularly preferably, the arched contact surface has the same radius or radius of curvature as the part-circle of the toothing.

Tightening elements which extend in the axial direction of the shank are preferably provided, in order to hold the individual shank sections with their toothings or friction surfaces in engagement or bearing contact. These tightening elements for example can be tightening or tension cables which are biased via spring elements such that the shank sections are always held in contact even with pivot movements, so that toothings remain meshingly engaged or friction surfaces are frictionally held in contact, so that a rolling takes place without any slip. The shank sections can also be held in contact via the subsequently described pull elements.

The deflection of the individual shank sections is preferably effected via pull elements, in particular via pull wires or pull cables. For this, preferably at least two pull wires extending in the longitudinal direction of the shank are provided, and these with their distal end are fastened on two diametrically opposite sides of the distal shank section. These two diametrically opposite sides, on which the pull wires are fastened and situated, are thereby located preferably in the pivot plane, within which the respective shank sections are to be pivoted to one another. The deflection of the shank in the radial direction, at which the shortening pull wire is situated, is then effected by way of tension on one of the pull wires. A deflection in the opposite direction is effected with pull on the other pull wire. The respective second pull wire is accordingly loosened or deflected. This deflection via pull wires corresponds essentially to the deflection which is known from flexible endoscopes.

It is further preferable for at least four pull wires to be provided, which are each fastened on a distal shank section in a manner offset by 90° with respect to the longitudinal axis, in order to permit a deflection in two different spatial directions, in particular in ones offset by 90° to one another. Thereby, two of the pull wires in a diametrically opposite manner preferably lie in the first described pivot plane and the two other pull wires in a diametrically opposed manner in a pivot plane which is offset by 90° about the longitudinal axis of the shank. I.e. two of the pull wires are responsible for the pivot movement in the first pivot plane and two of the pull wires are responsible for the pivot movement in the second pivot plane. If several shank sections which are movable to one another and which are alternately movable to one another in the two different planes are arranged in the shank, then a pivot movement in each case in only each second pivot joint and permitting a pivot movement in this pivot plane is effected with the actuation of the first pair of pull wires. A pivot movement in the normally directed pivot plane in the respective other pivot joints, is then effected on actuating the other pair of pull wires.

Preferably, four pull wires are provided and these are connected to one another at their distal ends. The connection of the pull wires simplifies the assembly. In particular, all pull wires or control cables are connected to one another in a distal shank section, i.e. the last movable shank section at the distal end. Thus, the pull wires departing from the distal end can be simply threaded through the envisaged guides and recesses of the proximally connecting shank sections, up to the proximal end. Preferably, the four pull wires can thereby be designed as two U-shaped loops which cross and are connected to one another in their crossing point or middle point. This connection for example can be effected by way of welding or bonding or clamping.

If the pull wires are not articulated on the distal shank section, but on shank sections which are situated further proximally, then it is also possible to move individual shank sections to one another in a defined manner via corresponding pairs of pull wires. I.e. a pair of pull wires is provided for each joint or each movable shank section, and this pair is fastened in the pivot plane of this shank section on diametrically opposed sides of the shank section. A defined serpentine deflection of the instrument can therefore be achieved.

Further preferably, guides for these pull wires are formed in those shank sections, which are situated proximally of the distal shank section or that shank section, on which the respective pull wires are fastened. Thus, one succeeds in the pull wires being led in the shank wall or on the shank wall in a defined manner. Moreover, the guides provide contact points which permit the deflection in a defined manner by way of tension on the pull wires in the first place. Thus, force engagement points are created for the pull wires.

According to a further preferred embodiment, the at least two shank sections which are movable adjacently one another comprise a side guide. I.e. the two shank sections are engaged with one another via a side guide extending parallel to the pivot plane. The side guide can be formed by one or more guide surfaces. Preferably, at least one web projects from one of the shank sections and engages into an opposite groove. Thereby, the web and the groove extend in the pivot plane or parallel to the pivot plane. According to a first preferred embodiment, only one side guide is provided in the form of a web which is preferably situated in the middle region. According to an alternative embodiment, two side guides which are distanced to one another in the diameter direction and which extend parallel to one another can also be provided.

As specified above, the shank of the medical instrument according to a preferred embodiment is designed in a hollow manner. For this, the at least two movable shank sections in each case can comprise a central cavity extending through the shank section in the axial direction, wherein the cavities of the several shank sections are aligned with one another. A continuous cavity extending in the axial direction is created through the movable shank sections in this manner. This cavity or channel permits the guiding and optional fastening of further subassemblies such as fiber optics, picture leads, electrical leads, flexible tubing or pipes for leading media such as gas or liquids, flexible tubing or pipes for leading application tools such as forceps, baskets, laser fibers, force transmission elements such as rods, cables, micro-drives such as piezo-drives, actuators based on shape memory, electroactive polymer actuators, dielectric elastomer actuators, magnetostrictive drives, magneto-rheological drives, pneumatic and hydraulic drives as well as electromagnetic drives etc.

The invention is hereinafter described by way of example and by way of the attached Figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic perspective total view of the distal end of a medical instrument;

FIG. 2 is a view according to FIG. 1 without the enveloping protective hose;

FIG. 3 is a schematic view showing the shank construction of the instrument according to FIGS. 1 and 2;

FIG. 4 is a schematic view showing the bending of the shank according to FIGS. 1-3;

FIG. 5 is a schematic lateral view of a second embodiment of the invention;

FIG. 6 is a view showing the bending of the shank according to FIG. 5;

FIG. 7 is a schematic view showing the construction of a shank according to a third embodiment of the invention;

FIG. 8 is a perspective view showing a shank section according to a further embodiment of the invention;

FIG. 9 is a perspective view showing a shank section of a further embodiment of the invention;

FIG. 10 is a schematic view showing the bending of two movable shank sections;

FIG. 11 is a schematic perspective view showing a serially bendable shank according to the invention;

FIG. 12 is a lateral view of the shank according to FIG. 11;

FIG. 13 is a schematic view showing a shank bendable in two directions;

FIG. 14 is a schematic view showing the construction of a shank according to a further embodiment of the invention;

FIG. 15 is a detailed view of the shank according to FIG. 14, without the distal shank section;

FIG. 16A is one of two opposite plan views of the central movable shank section of the embodiment according to FIGS. 14 and 15;

FIG. 16B is a lateral view showing the central movable shank section of the embodiment according to FIGS. 14 and 15;

FIG. 16C is the other of two opposite plan views showing the central movable shank section of the embodiment according to FIGS. 14 and 15;

FIG. 17 is a schematic view showing an end region of a shank section according to a further embodiment of the invention;

FIG. 18 is an enlargement of the detail XVIII in FIG. 17;

FIG. 19 is a schematic perspective view showing the abutment of two shank sections according to FIGS. 17 and 18;

FIG. 20 is a sectioned view of the arrangement in FIG. 19;

FIG. 21 is a schematic view showing an end region of a shank section according to a further embodiment of the invention;

FIG. 22 is an enlarged view showing the detail XXII in FIG. 21;

FIG. 23 is a schematic perspective view showing the installation of two shank sections according to FIGS. 21 and 22;

FIG. 24 is a plan view of the arrangement according to FIG. 23; and

FIG. 25 is a sectioned view along the line XXV-XXV in FIG. 24.

### DESCRIPTION OF THE PREFERRED EMBODIMENTS

The medical instrument according to the invention is preferably an endoscopic instrument and in the known manner comprises a shank 2, whose distal shank region 4 is designed in a bendable manner. A forceps jaw 6 is arranged at the distal end of the shank. The forceps jaw 6 in the known manner can be moved from the proximal end of the shank 2 (the proximal end is not shown here) via a handle or electrical drive. Actuation elements such as pull or push elements can run in the axial direction X in the inside of the shank 2 for this.

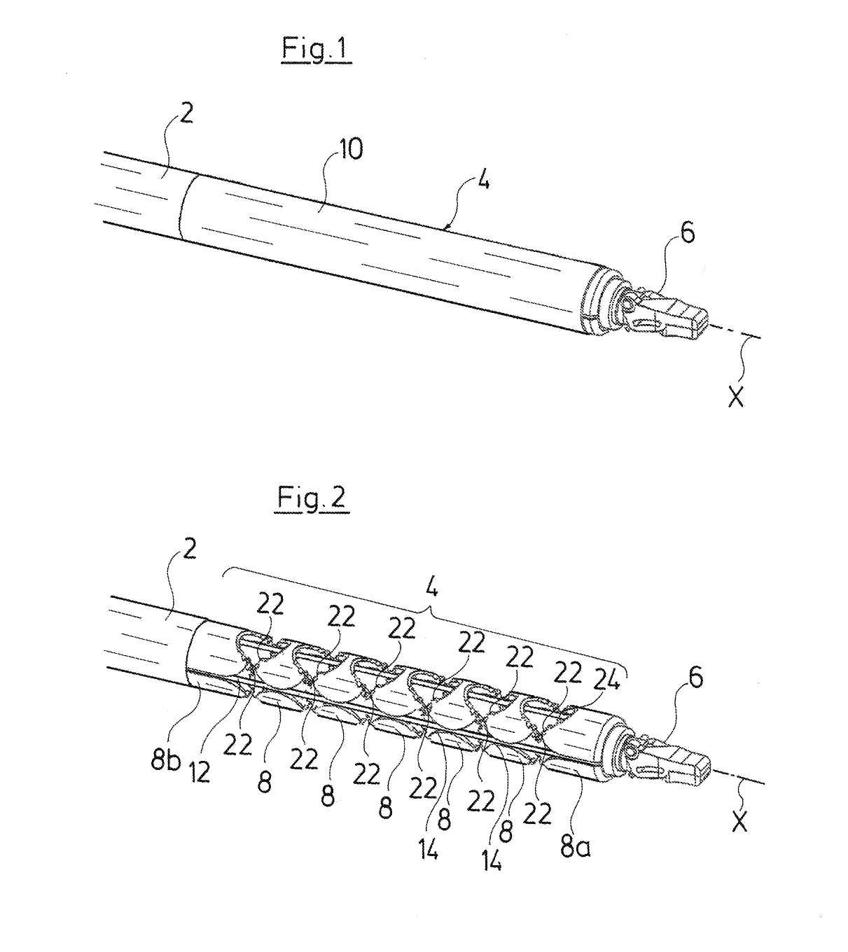

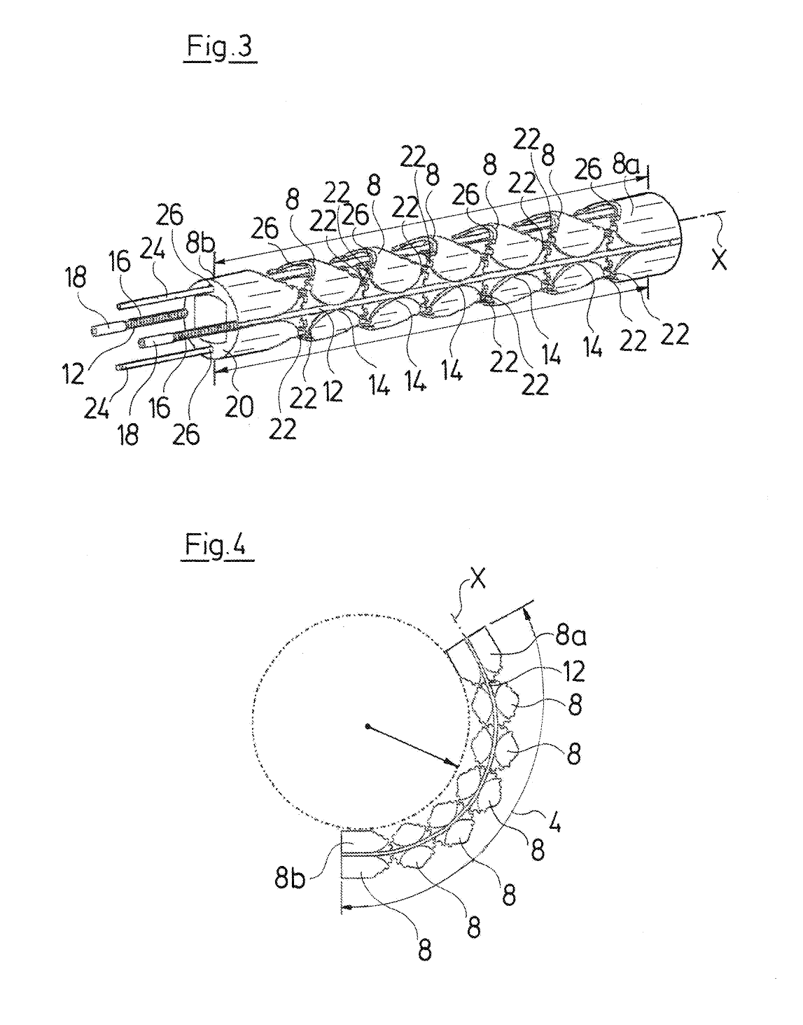

The distal shank region 4 consists of several shank sections 8 which are arranged axially adjacent one another. The shank sections 8 at their outer periphery are surrounded by a protective hose, as is schematically shown in FIG. 1. The shank sections 8 in the axial direction are held in a manner bearing on one another via two diametrically distanced tension cables 12. For this, the tension cables 12, as is shown in FIGS. 2 and 3, extend through guide grooves 14 in the walls of the shank sections 8. The tension cables 12 are fixed on the distal shank section 8a which is situated closes to the forceps jaw 6. Compression springs 16 are arranged at the opposite axial end on tension rods or tension cables 12, and with an axial end are supported on a sleeve 18 fixed on the tension cable 12 and with the opposite axial end are supported on the proximal end surface 20 of the proximal shank section 8b of the distal shank region 4. The compression springs 16 have the effect that the shank sections 8 on the tension rods or tension cables 12 are pressed to bear on one another.

The individual shank sections 8 are engaged with one another via toothings 22. The toothings 22 are formed on the individual shank elements 8 on two diametrically opposite sides and specifically at the sides, on which the guide grooves 14 through which the tension cables 12 extend are also situated. The toothings 22 are each designed in an arched manner, so that they form the section of a cog. The axes, about which the toothings 22 are curved in an arched manner, extend normally to the longitudinal axis X of the shank 2 in the diametrical direction through the shank sections 8 and thus cross the tension cables 12. The toothings 22 permit the shank sections 8 which are adjacent one another or neighbor one another, to be able to roll on one another via the toothings 22. The shank region 4 can thus be arcuate as is shown in FIG. 4.

The curvature is achieved by two pull cables 24 which extend in axial direction through guides in the form of holes 26 in the walls of the shank sections 8. The two pull cables 24 are arranged on two diametrically opposite sides of the shank sections 8 in a manner offset by 90° to the sides, on which the tension cables 12 are arranged. A diametrical connection line between the pull cables 24 thus extends normally to the axes, about which the toothings 22 are arcuate. The pull cables 24 are likewise fixedly connected on the distal-side shank section 8a which is situated closest to the forceps jaw 6 and thus to the distal end of the shank 2. They extend through the shank 2 up to the proximal end which is not shown here, and there are connected to drive elements or actuation elements in the known manner. The shank region 4 can be deflected into a direction depending on which of the pull cables 24 is pulled in the proximal direction and thus is shortened in the shank region 4. The pull cable 24 which is situated diametrically opposite is accordingly lengthened. A deflection in the other direction is effected accordingly by way of shortening the other pull cable 24. The pull cables 24 could also be designed as pull-push elements. One could then make do without one of the pull cables. A deflection in the one direction in this case could then be effected by way of pushing, and the deflection in the opposite direction by way of pulling the pull-push element.

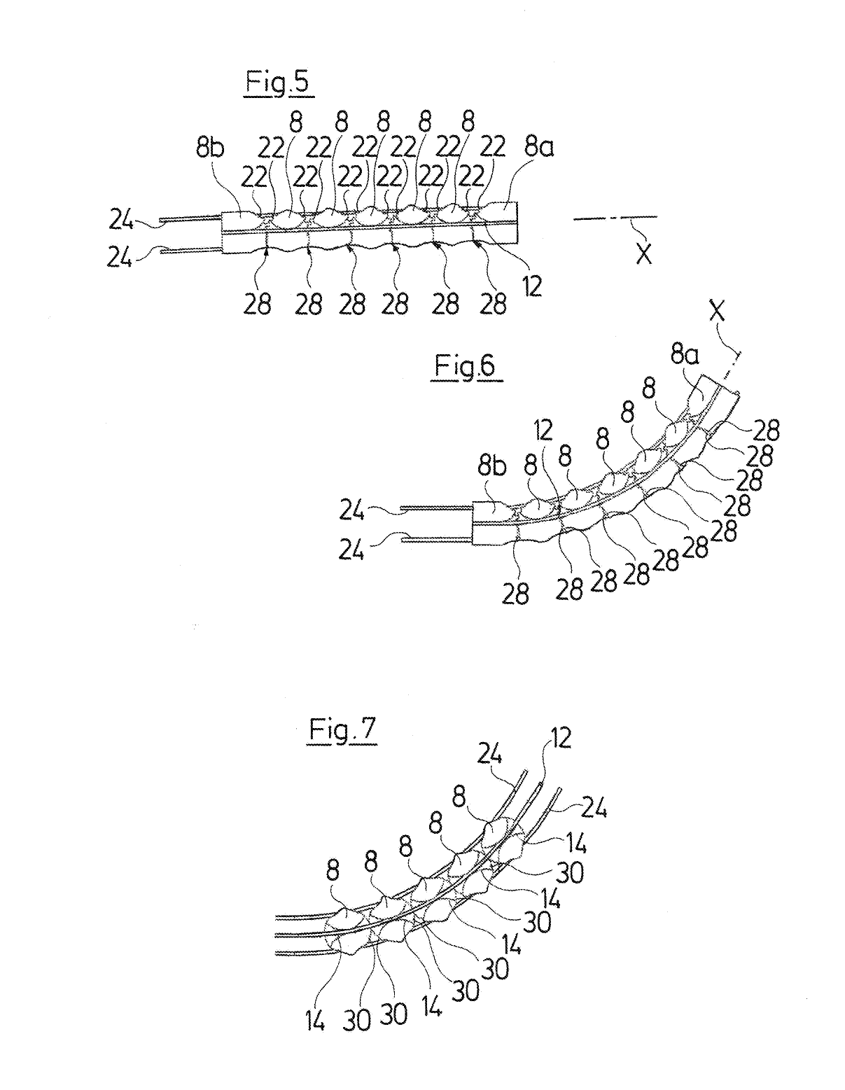

An embodiment, with which a deflection is possible in only one direction, is also conceivable. Such an embodiment is shown in FIG. 5. There, the toothings 22 extends essentially only in 90° arcs and not in a semicircular manner, as shown in the previous figures. The toothings thus only extend on one side of the tension cables 12. Shoulders 28 which extend essentially normally to the longitudinal axis X and which, with the axial extension shown in FIG. 5, already bear on one another are formed on the other side, on the shank sections 8. I.e. with the embodiment example according to FIG. 5, a deflection can only be effected upwards in FIG. 5, by way of shortening the upper pull cable 24, whereas the lower pull cable 24 is lengthened in FIG. 5. An extension in the straight direction is then effected by way of pulling on the lower pull cable 24. The respectively arcuate or deflected condition is shown in FIG. 6. Gaps then form between the shoulders 28 on arching.

A further embodiment example, with which the shank sections 8 have no toothings but frictionally bear on one another via friction surfaces 30 is shown in FIG. 7. The construction otherwise corresponds to that which has been described previously, i.e. here too the shank sections 8 are held in contact by way of tension rods or tension cables 12. Here too, the adjacent shank sections 8 roll on one another given a deflection or bending-out, as has been described previously with regard to the toothings 22. The friction surfaces 30 are thereby arcuate in the manner of a circular arc, in a manner corresponding to the previously described toothings 22, and with regard to their surface nature are designed such that a slip or a slipping between the shank sections 8 is prevented. Thus, a geometrically defined deflection of the shank is achieved also with this embodiment, as with the previously described embodiments with the toothings.

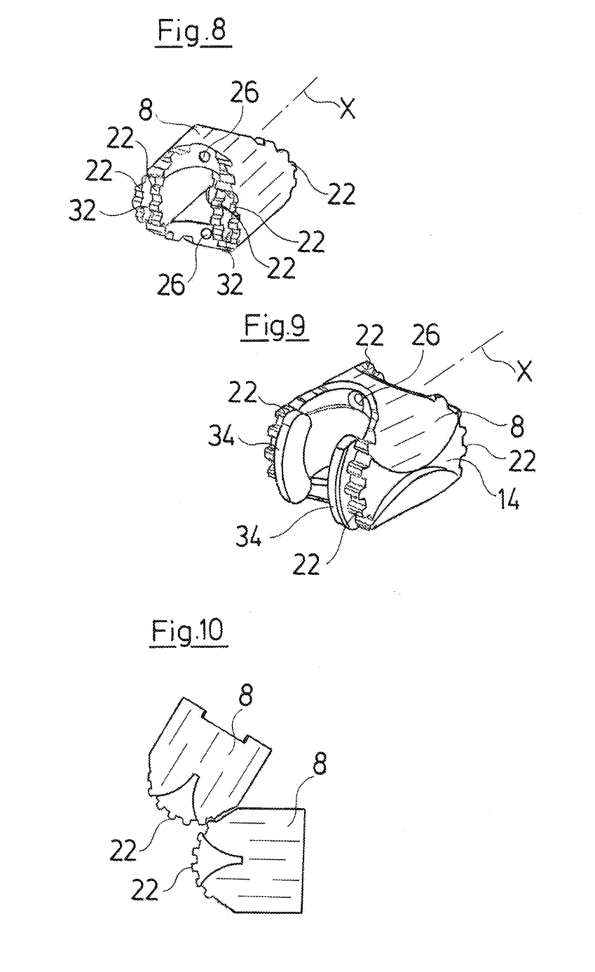

FIG. 8 by way of example of an individual shank section 8 shows a different type of guiding for the tension cables 12. No guide grooves 14 are provided with this embodiment, but guide channels 32 extending through the walls in the axial direction X are provided in the inside of the side walls. Here, the guide channels 32 extend through the peripheral wall sections, on whose axial ends the toothings 22 are formed. I.e. here an opening to the guide channels 32 is formed in the middle region of the toothings 22. Holes 26, through which the pull cables 24 are led are also recognizable in FIG. 8.

FIG. 9 shows a further variant of the construction of an individual shank section 8, with which guide surfaces 34 projecting in the axial direction X beyond the axial face edges of the toothings 22 are formed on the inner periphery, laterally of the toothings 22. The guide surfaces 34 are only formed at one axial end of the shank sections 8, so that at an adjacent shank section 8 they can engage from the opposite axial end, on which no guide surfaces 34 are formed, into the inside of the shank section 8 and come to bear on the inner periphery laterally of the toothings 22. The guide surfaces 34 in this manner form a lateral guide for the adjacent shank sections 8, so that these cannot move to one another in a direction of the curvature axes of the toothings 22 transverse to the longitudinal axis X.

FIG. 10 shows a minimal configuration of a bendable shank region which is formed from two shanks sections 8 which are adjacent one another and pivotable to one another. These shank sections 8 are engaged with one another via toothings 22. It is to be recognized that an angling or bending of 120° can be achieved with this design, which permits a rolling of the shank sections 8 via their toothings 22. The arrangement of more shank sections 8 as is shown for example in FIG. 4 permits a similar angle with a larger radius.

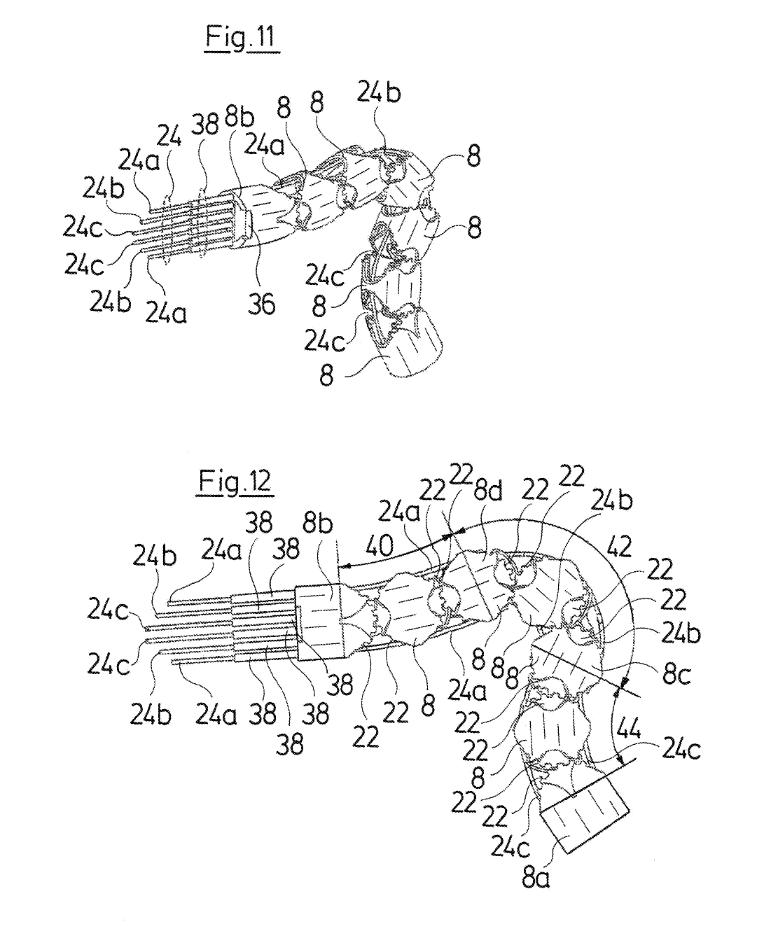

FIGS. 11 and 12 show a further preferred embodiment, with which the shank region can be bent or angled in a sequential manner. Here too, the movable or bendable shank region 4 is formed from several shank sections 8 which are axially adjacent one another and are held together by way of non-shown tension cables led through axial holes 36 in the walls of the shank sections 8. The holes 36 extend axially through the walls of the shank sections 8 on two diametrically opposite sides, similarly to the guide channels 32 in FIG. 8. The embodiment according to FIGS. 11 and 12 differs from the previously described embodiments in that here three pairs of pull cables 24a, 24b and 24c are present. The pull cables 24 are in each case led in a sleeve 38 in the manner of a Bowden cable. The sleeve serves for supporting the pull cables 24 on the proximally first shank section 8b. The sleeves 38 are fixed in the feed-throughs in the proximal shank sections 8d for this.

Three bend regions 40, 42 and 44 can be realized in the shank region 4 by way of the three pairs of pull cables 24a, 24b and 24c. The pull cables 24c are fixed on two diametrically opposite sides of the distal shank section 8a, as the pull cables 24 according to the preceding embodiment examples. The sleeves 38 of the pull cables 24c are simultaneously fixed in the shank sections 8c which forms the transition between the bend regions 42 and 44. The pull cables 24b with their distal ends are fixed in the shank section 8c, whereas the sleeves 38 of these two pull cables 24c are supported on the shank section 8d which forms the transition between the bend regions 40 and 42. The pull cables 24a with their distal ends are fastened on the shank section 8d and the sleeves 38 of the pull cables 24a are fixed in the shank section 8d. The bend regions 40, 42, and 44 can be individually deflected by way of this design. In FIG. 12, the upper pull cable 24a is shortened and simultaneously the lower pull cable 24a is lengthened, in order to achieve the deflection shown in the FIGS. 11 and 12. The first bend region 40 between the shank sections 8a and 8b is curved upwards by way of this. The shank sections 8 and their toothings 22 thereby roll on one another, as described above. The lower of the pull cables 24b in FIG. 12 is simultaneously shortened and the upper pull cable 24b is lengthened, so that the second bend region 42 is curved downwards, wherein the shank sections 8c and 8d as well as the further shank section 8 situated between these roll on one another via their toothings 22. In FIG. 12, the upper pull cable 24c in turn is shortened, and the lower pull cable 24c is lengthened, for the curvature of the third bend region 44, so that the third bend region 44 is curved to the right or to upwards in FIG. 12. Thereby, the shank sections 8c and 8a roll with their toothings 22 on the intermediately lying shank section 8. It is to be understood that one can also create more than three individually bendable shank regions by way of the arrangement of correspondingly more sections 8 and individual pull cables 24. It is also conceivable to provide only two bend regions.

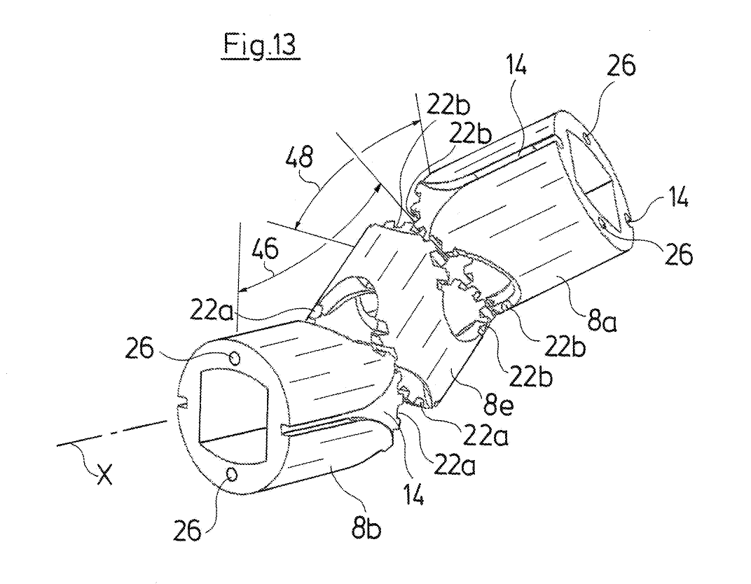

FIG. 13 now shows an embodiment, with which the shank region 4 can be bent in two different directions. Only three shank sections 8a and 8b as well as an intermediately lying shank section 8e are shown with this embodiment example. With this embodiment, the toothings 22a between the shank sections 8b and 8e are arcuate in a first direction, i.e. curved about axes which are directed in a first diameter direction with regard to the longitudinal axis X of the shank. The toothings 22b between the shank sections 8a and 8e which are likewise arched, are curved in a second direction, i.e. each curved about an axis which extends normally to the curvature axes of the toothings 22a in the diametric direction to the longitudinal axis X of the shank. The sections 8e and 8b are thus movable to one another in a first pivot plane, whereas the shank sections 8a and 8e are movable to one another in a second pivot plane 48 which extends at right angles to the first pivot plane 46. The pivot movements are initiated by pull cables 24 which in the manner described by way of FIGS. 11 and 12 can be fastened sequentially on individual shank sections 8 or however can all be fastened on the distal shank section 8a, so that the shaft, although being able to be bent in two directions, however only in an angular range. The pull cables 24 and the tension cables 12 extend through the guides 14 and the holes 26 as well as suitable holes or guide channels in the shank section 8e, which are not shown here. Moreover, it is to be understood that the bend region with the embodiment according to FIG. 13 can also be designed longer, by way of several shank elements 8e with toothings 22a and 22b rotated to one another being able to be arranged between the distal shank section 8a and the proximal shank section 8b. Thereby, the individual shank sections 8e which are adjacent one another, are in each case rotated by 90° to one another about the longitudinal axis X.

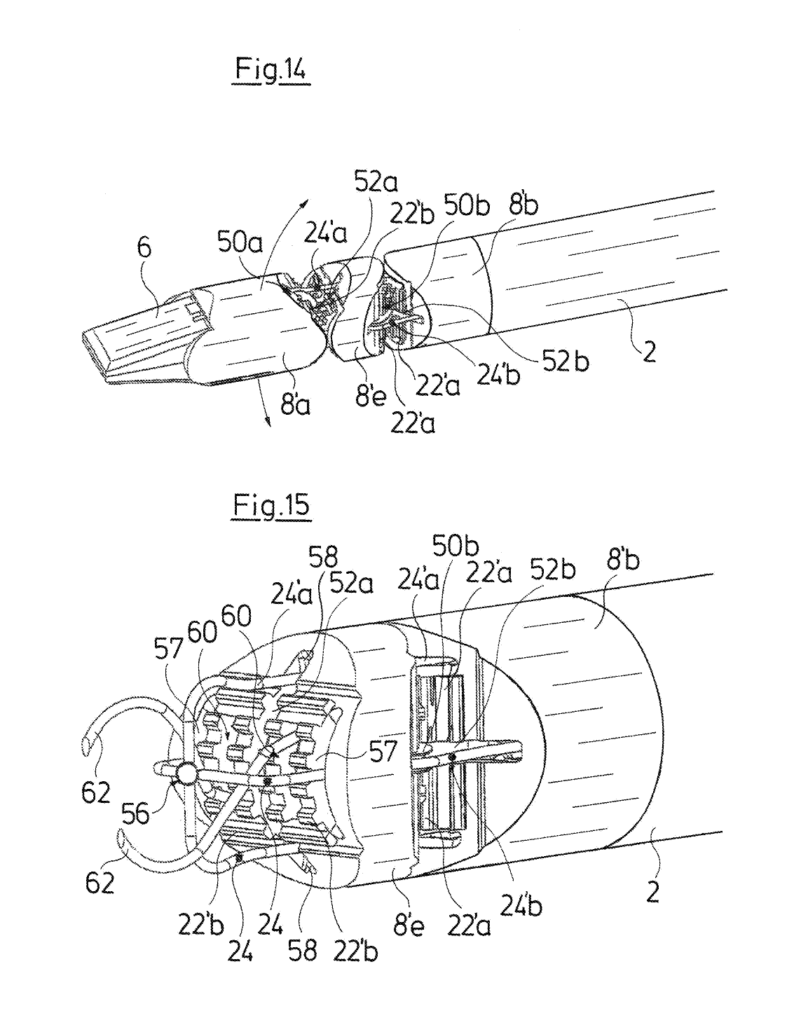

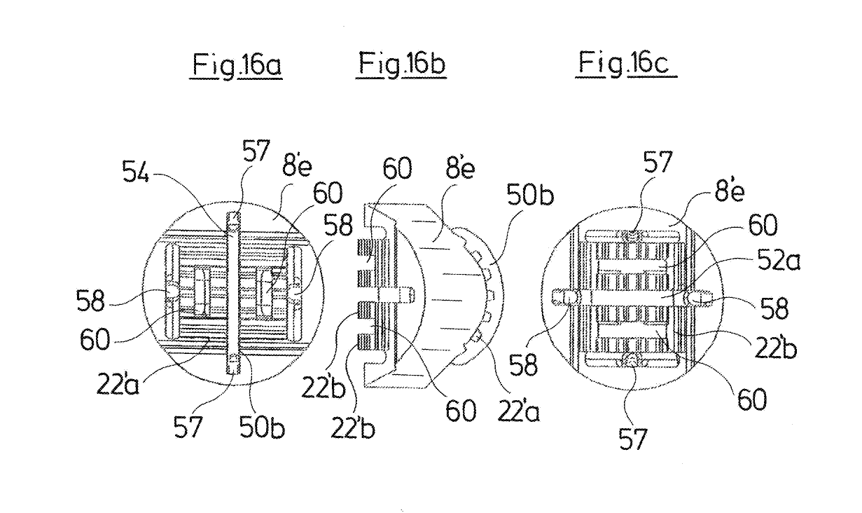

A further embodiment of the invention is described by way of FIGS. 14-16. The shank 2 which is shown in FIG. 14 comprises a forceps jaw 6 at its distal end. Thereby, the forceps jaw 6 is arranged on a distally movable shank section 8′a. This shank section 8′a is connected via an intermediately mounted further movable shank section 8′e to a shank section 8′d which is fixedly arranged on the rigid part of the shank 2. The shank sections 8′b and 8′e roll on one another via toothings 22′a. The shank sections 8′e and 8′a roll on one another via toothings 22′b. Thereby, the toothings 22′a and 22′b are arranged in two planes which are rotated to one another by 90° about the longitudinal axis of the shank 2, so that the shank section 8′a is angled or bent with respect to the shank section 8′e in a plane which is offset by 90° to the plane, in which the which the shank section 8′e is bent with respect to the shank section 8′b. The toothings 22′b and 22′a here extend essentially over the whole central region of the shank sections 8′, and only broken through by openings.

With this embodiment, in particular a central side guide is provided which is formed by projecting webs 50a and 50b. The web 50a extends in an arched manner at the proximal end of the shank section 8′a parallel to the pivot plane between the shank section 8′a and the shank section 8′e. The web 50b departing form the proximal side of the shank section 8′e, facing the shank section 8′b, extends parallel to the pivot plane between the shank sections 8′e and 8′b. The web 50a engages into a recess or groove 52a, which is formed on the distal side of the shank section 8′e which faces the shank section 8′a. Accordingly, the web 50b engages into a groove 52b which is formed on the distal side of the shank section 8′b which faces the shank section 8′e. The groove 52a thereby extends parallel to the web 50a and the groove 52b extends parallel to the web 50b. A side guidance transverse to the respective pivot plane is realized by way of the engagement of the webs 50 into the associated grooves 52.

The projections or webs 50a and 50b simultaneously serve for guiding the pull cables 24′a and 24′b. For this, the webs 50a and 50b on their outer or upper surfaces are provided with channel-like recesses 54 which form cable guides. Here, two pairs of pull cables 24′a and 24′b are provided, wherein the pair of pull cables 24′b serve for deflecting the shank section 8′e with respect to the shank section 8′b, and the pair of pull cables 24′a serves for deflecting the shank section 8′a with respect to the shank section 8′e. The two pairs of pull cables 24′ are fixedly connected to one another in the distal shank section 8′a at the connection point 56. This simplifies the assembly, since the pull cables 24′ can be threaded from the distal end through all shank sections 8. The pull cables 24′b extend through holes 57 laterally of the web 50b, whereas the pull cables 24′a extend through the holes 58 on the longitudinal ends of the groove 52a. The holes 57 and 58 extend in the axial direction through the shank section 8′e, wherein the holes 58′ widen towards the proximal end and the holes 57 towards the distal end. Moreover, lying further to the inside, holes 60 are formed in the middle shank section 8′e, through which holes pull cables or control cables 62 forming actuating cables for opening and closing the forceps jaw 6 extend. The holes 60 form guides for the pull cables 62, so that the pull cables 62 extend in the inside of the shank in the central region through this in the axial direction.

With regard to the previously described embodiments, it is to be understood that a design with friction surfaces, as is shown in FIG. 7, can also be applied alternatively with all shown embodiment examples.

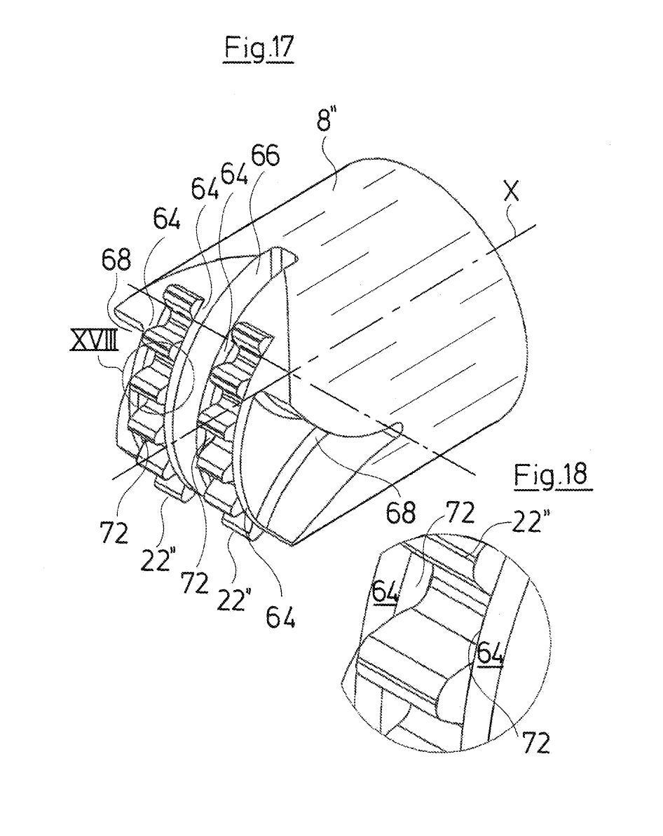

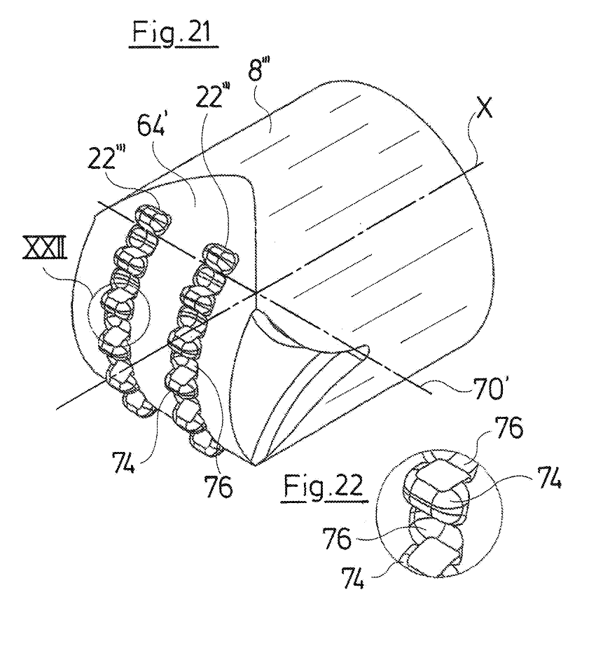

Apart from a toothing, a contact via contact surfaces of two adjacent shank sections is also given with the embodiments shown in FIGS. 17 to 20 as well as FIGS. 21 to 25. Two arched toothings 22′ are arranged at the end of the shank section 8″ with the embodiment according to FIGS. 17 to 20. Arched contact surfaces 64 extend parallel to these arched toothings 22″, on both sides of each toothing 22″. The contact surfaces 64 extend in the radial direction between the tip circle and the root circle of the toothings 22′, preferably on the part circle. Thus, the heads of the teeth of the toothings 22′ project radially outwards beyond the contact surface 64, so that the side surfaces of the teeth can come into contact with the side flanks 72 on the opposite toothing of an adjacent shank section, for limiting the lateral movement. A groove or a slot 66, through which pull cables can extend as described above with regard to the groove 52a and the holes 58, extends between the inner contact surfaces 64 between the two toothings 22′. Accordingly, grooves or recesses 68, through which the tension cables or pull cables as have been described above as tension cables 12 and pull cables 24 and 24′ can extend, are provided on the outer sides of the outer contact surfaces 64, laterally of the toothings 22.

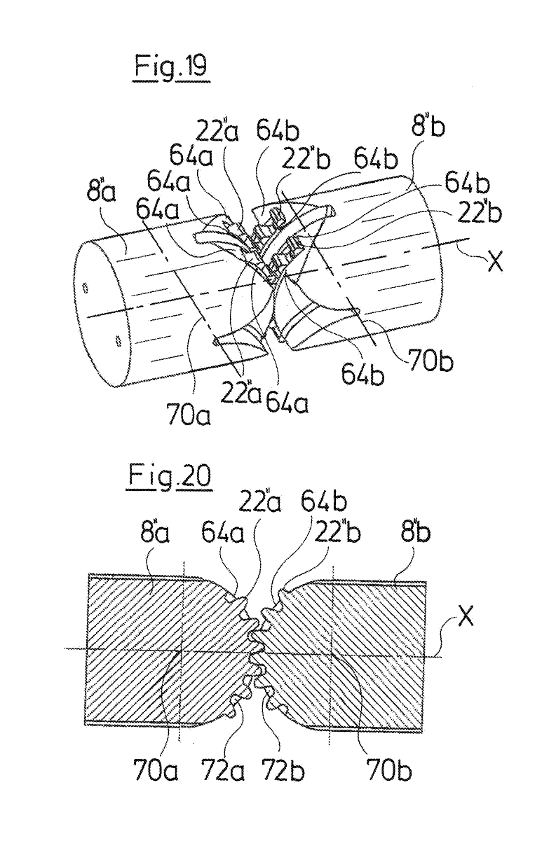

If two of the shank sections, as are shown in FIGS. 17 and 18, are arranged lying opposite one another as is shown in FIG. 19, then the toothings 22″a and 22″b which lie opposite one another mesh into one another as described above by way of the other embodiments. The contact surfaces 64a and 64b lying opposite one another simultaneously bear on one another. The contact surfaces 64a and 64b thus roll on one another given a bending. The toothings 22″a and 22″b simultaneously roll on one another in a meshing manner. A positive fit ensuring a defined rolling without slip is ensured by way of the toothings 22″. The contact surfaces 64 thereby keep the toothings 22″a and 22″b meshing into one another at a defined distance to one another which means that the rotation or pivot axes 70a and 70b which form the middle point of the arch, along which the toothings 22″a and 22″b extend, are held at a defined distance to one another.

The side flanks 72 (72a, 72b) which extend normally to the contact surfaces 64 and face the toothings 22″ form a lateral guide which prevents a displacement of the two shank sections 8″ and 8″b along the pivot axes 70a and 70b. The side surfaces of the teeth of the toothings 22″a and 22″b abut onto these inner side flanks 72 of the contact surfaces 64 and thus form a stop limiting the lateral movement.

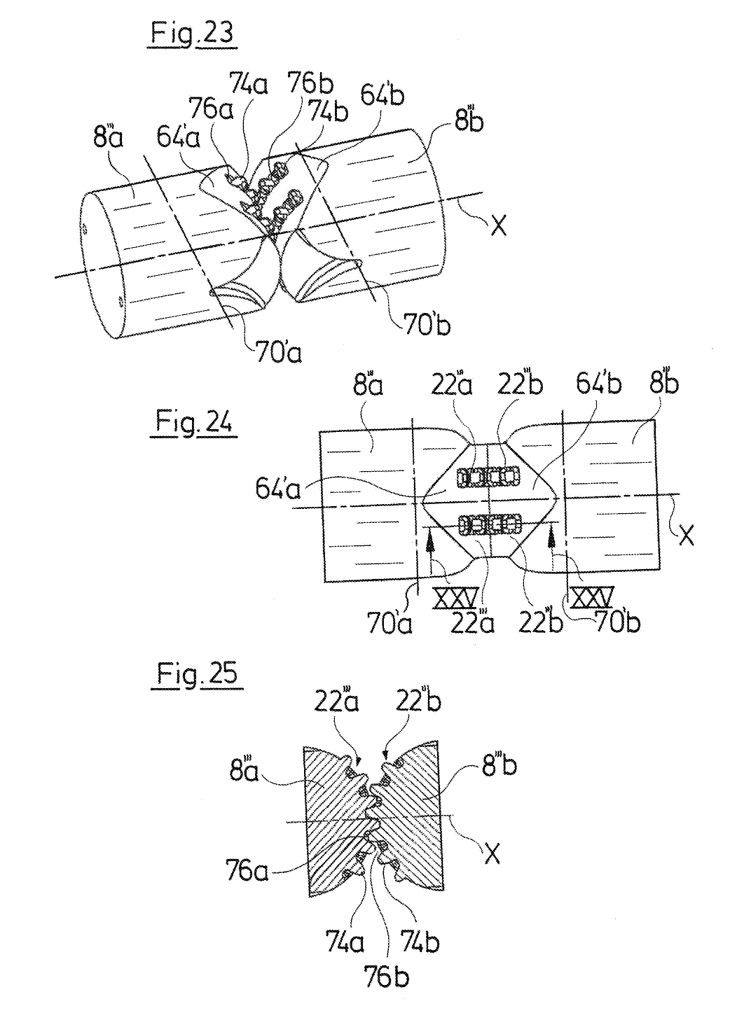

FIGS. 21 to 25 show a further embodiment of the invention similar to the previously described embodiment. With this embodiment, the end region of the shank section 8′ comprises an arched contact surface 64′ which is arcuate in the manner of a circular arc about the pivot axis 70′. Two arched toothings 22′ which are parallel to one another are formed in this contact surface 64′. The toothings are formed by way of alternating teeth 74 and troughs 76. The shape of the troughs 76 is thereby adapted to the shape of the teeth 74, so that the teeth 74a of a first shank section 8′a can engage or mesh into the opposite troughs 76b of an adjacent or opposite second shank section rb, and vice versa, the teeth 74b of the second shank section 8′″b mesh into the troughs 76a of the first shank section 8′″a, as is shown in FIGS. 23 to 25. With this embodiment too, the two shank sections 8′″a and 8′″b roll on one another via their contact surfaces 64′a and 64′b, whereas the toothings 22′″a and 22′″b which are formed by the teeth 74 and troughs 76 in the previously described manner, meshingly engage into one another. A positive, slip-preventing engagement is therefore realized by the toothings 22′″, whereas the contact of the contact surfaces 64′ holds the pivot axes 70a′ and 70b′ of the shank sections 8′″a and 8′″b at a defined distance to one another, so that the toothings 22′″a and 22′″b can mesh into one another in the desired manner, without blocking.

The teeth 74 and troughs 76 are designed such that their sides situated in the direction of the pivot axes 70′ are designed in a rounded manner. A certain centring in the direction of the pivot axes 70′ on engagement of the teeth 74 into the troughs 76 is achieved by way of this. With this embodiment too, the arched contact surface 64′ preferably extends on the part-circle of the thus formed toothings 22′″.

It is to be understood that with the embodiments according to FIGS. 17 to 25, the guiding of the pull and tension cables can be effected in a manner described in the preceding embodiments. The arrangement of several shank sections can also be effected in the manner as has been described above. Alternating pivot directions, as have been described by way of FIGS. 13 to 16, are also possible with these embodiments, by way of the pivot axes or curvature axes, about which the contact surfaces 64, 64′ and toothings 22″ and 22′″ pivot, being arranged between individual shank sections in each case offset to one another by a certain angle, preferably 90°. The above description is referred to as far as this is concerned.

While specific embodiments of the invention have been shown and described in detail to illustrate the application of the principles of the invention, it will be understood that the invention may be embodied otherwise without departing from such principles.

1. A medical instrument comprising:
a shank which comprises at least two adjacent shank sections which are movable relative to one another, wherein the at least two movable shank sections each comprise toothings or friction surfaces and frictionally roll on one another via the friction surfaces or are meshingly engaged with one another via the toothings.

2. A medical instrument according to claim 1, wherein the shank further comprises additional shank sections to provide a plurality of shank sections which can be moved relative to one another and of which in each case two shank sections which are adjacent to one another are meshingly engaged with one another via toothings or friction surfaces.

3. A medical instrument according to claim 1, wherein two adjacent shank sections at their face ends which lie opposite one another are provided with corresponding toothings or friction surfaces.

4. A medical instrument according to claim 1, wherein the toothings or friction surfaces are designed in an arched manner.

5. A medical instrument according to claim 4, wherein the toothings which mesh with one another or frictions surfaces have the same radius of curvature on the face ends of two adjacent shank sections which lie opposite one another.

6. A medical instrument according to claim 4, wherein the toothings which mesh with one another or frictions surfaces are arcuate in opposite directions at the face ends of two adjacent shank sections which lie opposite one another.

7. A medical instrument according to claim 1, wherein the adjacent shank sections have face ends which face one another that comprise a toothing or friction surfaces, in each case on two sides which are diametrically opposite.

8. A medical instrument according to claim 7, wherein the toothings or friction surfaces extend in an arched manner about the same axis, on the two diametrically opposed sides.

9. A medical instrument according to claim 1, wherein the shank comprises at least three shank sections which can be moved relative to one another, of which the respective adjacent shank sections are meshingly engaged with one another via the toothings or friction surfaces, wherein first and second shank section are movable relative to one another in a first pivot plane and the second and the third shank section in a second pivot plane.

10. A medical instrument according to claim 9, wherein the first and the second pivot plane extend normally to one another.

11. A medical instrument according to claim 9, wherein the toothings or friction surfaces are designed in an arched manner, and the toothings or friction surfaces between the first and the second shank section are arcuate about axes which extend normally to the axes, about which the toothings or friction surfaces between the second and the third shank section are actuate.

12. A medical instrument according to claim 1, wherein the at least two movable shank sections are meshingly engaged with one another via toothings and simultaneously roll on one another via arched contact surfaces.

13. A medical instrument according to claim 12, wherein each shank section comprises at least one arched contact surface and a toothing, wherein the arched contact surface is arcuate parallel to the toothing.

14. A medical instrument according to claim 13, wherein the arched contact surface has the same radius as the part-circle of the toothing.

15. A medical instrument according to claim 1, further comprising at least two pull wires extending in the longitudinal direction of the shank and having distal ends fastened on two diametrically opposite sides of a distal shank section.

16. A medical instrument according to claim 15, wherein four pull wires are provided, which are fastened on a distal shank section in a manner offset in each case by 90° with respect to the longitudinal axis.

17. A medical instrument according to claim 15, wherein four pull wires are provided, which are connected to one another at their distal end.

18. A medical instrument according to claim 16, further comprising guides for the pull wires formed in shank sections which are situated proximally of the distal shank section, on which the pull wires are fastened.

19. A medical instrument according to claim 1, wherein the at least two adjacent shank sections are engaged with one another via at least one side guide which extends parallel to the pivot plane.

20. A medical instrument according to claim 1, wherein the at least two movable shank sections each comprise a central cavity which extends in the axial direction through the shank section, wherein the cavities of the at least two movable shank sections are aligned to one another.
